(19) United States
(12) Patent Application Publication
(10) Pub. No.: US 2025/0162967 A1
(43) Pub. Date: May 22, 2025

(54) PROCESS FOR PRODUCING A REFINED 1,4-BUTANEDIOL STREAM

(71) Applicant: JOHNSON MATTHEY DAVY TECHNOLOGIES LIMITED, London (GB)

(72) Inventors: Henry Arthur CLAXTON, London (GB); Paul GORDON, Stockton-On-Tess (GB); Graham REED, London (GB)

(21) Appl. No.: 18/839,203

(22) PCT Filed: Mar. 8, 2023
(86) PCT No.: PCT/GB2023/050543

§ 371 Date: Aug. 16, 2024

(30) Foreign Application Priority Data
Mar. 9, 2022 (GB) 2203264.3

(51) Int. Cl. C07C 29/88; C07C 29/149

(52) U.S. Cl. CPC C07C 29/88; C07C 29/149

(57) ABSTRACT

A process for producing a refined 1,4-butanediol stream is disclosed. The process comprises hydrogenolysis of dialkyl succinate in one or more mixed vapour/liquid phase reaction stages to form a crude 1,4-butanediol stream comprising 1,4-butanediol, γ-butyrolactone, tetrahydrofuran and alkanol and passing the crude 1,4-butanediol stream to a refining process, wherein at least some of the γ-butyrolactone, tetrahydrofuran and alkanol is removed from the 1,4-butanediol, and recovering from the refining process a refined 1,4-butanediol stream having a higher concentration of 1,4-butanediol than the crude 1,4-butanediol stream. The refining process comprises a polishing section in which an intermediate stream comprising 1,4-butanediol and 2-(4′-hydroxybutoxy)-tetrahydrofuran is passed over a catalytic bed to reduce the 2-(4′-hydroxy butoxy)-tetrahydrofuran content of the intermediate stream.

### FIELD OF THE INVENTION

The present invention relates to process for producing a refined 1,4-butanediol stream. In particular, but not exclusively, the present invention relates to a process for producing a refined 1,4-butanediol stream following hydrogenolysis of dialkyl succinate in one or more mixed vapour/liquid phase reaction stages.

### BACKGROUND

Butane-1,4-diol is used as a monomer in the production of plastics, such as polybutylene terephthalate, polybutylene succinate (PBS) and polybutylene adipate terephthalate (PBAT). It is also used as an intermediate for the manufacture of γ-butyrolactone and of the important solvent, tetrahydrofuran.

One route to butane-1,4-diol involves reaction of acetylene with formaldehyde by the Reppe reaction to yield butyne-1,4-diol which is then hydrogenated to produce butane-1,4-diol.

Another process for production of butane-1,4-diol uses maleic anhydride as a starting material. This is esterified with an alkanol, usually a C₁ to C₄ alkanol such as methanol or ethanol, to yield the corresponding dialkyl maleate which is then subjected to hydrogenation to dialkyl succinate and hydrogenolysis to yield butane-1,4-diol and the alkanol which can be recycled to produce further dialkyl maleate. Processes and plant for the production of dialkyl maleates from maleic anhydride are described, for example, in U.S. Pat. No. 4,795,824 and in WO90/08127. The vapour-phase hydrogenation of dialkyl maleates to yield butane-1,4-diol is discussed further in U.S. Pat. Nos. 4,584,419, 4,751,334, and WO88/00937.

In the hydrogenolysis of a dialkyl succinate, such as dimethyl succinate or diethyl succinate, there may also be produced amounts of the valuable by-products, γ-butyrolactone and tetrahydrofuran. Since there is a ready market for these by-products, their co-production with butane-1,4-diol is not disadvantageous. In addition, the hydrogenolysis product mixture will normally contain minor amounts of the corresponding dialkyl succinate, n-butanol, the corresponding dialkyl alkoxysuccinate, e.g. diethyl ethoxysuccinate, and water.

Another minor by-product has been identified as a cyclic acetal, i.e. 2-(4′-hydroxybutoxy)-tetrahydrofuran of the formula:

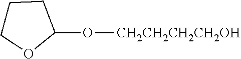

The cyclic acetal by-product, i.e. 2-(4′-hydroxybutoxy)-tetrahydrofuran, is troublesome because its boiling point lies very close to that of butane-1,4-diol and because it forms an azeotrope therewith. Hence it is difficult, if not impossible, to produce using conventional distillation techniques a butane-1,4-diol product which is essentially free from this cyclic acetal. Hence butane-1,4-diol produced by this hydrogenolysis route in the prior art typically are said to contain from about 0.15% by weight to about 0.20% by weight of the cyclic acetal with other impurities in total comprising no more than about 0.02% by weight. The presence of even minor traces of the cyclic acetal, 2-(4′-hydroxybutoxy)-tetrahydrofuran, in butane-1,4-diol is disadvantageous because it is a colour forming material and hence gives rise to colour formation in the butane-1,4-diol.

WO9736846A1, WO2006037957A1 and WO2013034881A1 describe methods for purifying butane-1,4-diol.

WO9736846A1 suggests that the cyclic acetal, 2-(4′-hydroxybutoxy)-tetrahydrofuran, may be formed by reaction of butane-1,4-diol with 4-hydroxybutyraldehyde which is a potential intermediate in the sequence of hydrogenolysis reactions or can be formed by dehydrogenation of butane-1,4-diol itself. WO9736846A1 then describes a process for the purification of a substantially anhydrous butane-1,4-diol feed containing a minor amount of the cyclic acetal, 2-(4′-hydroxybutoxy)-tetrahydrofuran, which comprises hydrogenating the butane-1,4-diol feed in a hydrogenation zone in the presence of a hydrogenation catalyst, and recovering from the hydrogenation zone a butane-1,4-diol product that has a reduced content of 2-(4′-hydroxybutoxy)tetrahydrofuran, characterised in that hydrogenation is effected in the presence of from about 0.5 by weight up to about 5% by weight, based upon the weight of the butane-1,4-diol feed, of water. In such a process the added amount of water may correspond to a water: 2-(4′-hydroxybutoxy)-tetrahydrofuran molar ratio of from about 20:1 to about 500:1.

WO2006037957A1 suggests that the cyclic acetal, 2-(4′-hydroxybutoxy)-tetrahydrofuran, may be formed by reaction of the 1,4-butanediol with 2-hydroxytetrahydrofuran which is a potential intermediate in the sequence of the hydrogenolysis reactions and/or it may be formed by the dehydrogenation of the 1,4-butanediol to hydroxybutyroaldehyde and cyclisation thereof to the more stable 2-hydroxytetrahydrofuran. WO2006037957A1 then describes a process for the purification of a crude liquid feed stream comprising 1,4-butanediol and a minor amount of 2-(4′-hydroxybutoxy)-tetrahydrofuran and/or precursors thereof wherein the process comprises passing the crude feed in the presence of hydrogen in a reaction zone over a heterogeneous liquid tolerant copper catalyst in the liquid phase at hydrogenation conditions and recovering a purified stream of 1,4-butanediol having a lower amount of 2-(4′-hydroxybutoxy)-tetrahydrofuran than the crude liquid feed stream.

WO2013034881A1 identifies an issue with the formation of 4-hydroxybutyl (4-hydroxybutyrate) in prior processes. The formation of 4-hydroxybutyl (4-hydroxybutyrate) is an equilibrium reaction in which the 4-hydroxybutyl (4-hydroxybutyrate) can revert to 1,4-butanediol and γ-butyrolactone under certain conditions. WO2013034881A1 identifies that in the distillation arrangements of the prior art these heavy components fractionate in the bottom of conventional or divided wall columns and, in the high temperature and high residence time regions of the column reboiler and sump, components such as 4-hydroxybutyl (4-hydroxybutyrate) react to reform to lighter components including γ-butyrolactone. That causes a problem with conventional distillation arrangements in that the light components, such as γ-butyrolactone, which are the result of the reaction in, for example, the sump, cannot be removed overhead from systems having conventional side draw arrangements. This is because the light components that are produced by the reaction of the heavy components in the column sump travel back up the column and contaminate the product side draw with light components and thereby limit the purity of the product that can be removed at the side draw. WO2013034881A1 also identifies that, in the hydrogenation of esters, such as those described in U.S. Pat. Nos. 4,584,419, 4,751,334 and WO88/00937, 3-(4-hydroxybutoxy)-tetrahydrofuran is formed as an impurity, and that it will be understood that this is different from the 2-(4′-hydroxybutoxy)-tetrahydrofuran discussed above. WO2013034881 discloses that the presence of the additional γ-butyrolactone formed in the sump will render it more difficult to remove the 3-(4-hydroxybutoxy)-tetrahydrofuran in the final 1,4-butanediol distillation column and hence further limit the purity of the 1,4-butanediol available by conventional separation processes. WO2013034881A1 then discloses a process for purifying a stream comprising 1,4-butanediol comprising the steps of:
(a) supplying a crude product stream comprising 1,4-butandiol and one or more of γ-butyrolactone, 2-(4-hydroxybutoxy)-tetrahydrofuran, 4-hydroxybutyl (4-hydroxybutyrate), and 3-(4-hydroxybutoxy)-tetrahydrofuran to a first distillation column;
(b) removing a side-draw comprising 1,4-butanediol, and light components, said light components including at least some of those produced by reaction in the first distillation column;
(c) passing the stream to a hydrogenation zone;
(d) subjecting the stream from step (c) to hydrogenation in the hydrogenation zone in the presence of a hydrogenation catalyst, and recovering from the hydrogenation zone a 1,4-butanediol product stream having a reduced content of 2-(4-hydroxybutoxy)-tetrahydrofuran, and optionally additionally including (4-hydroxybutyl)-4-hydroxybutyrate formed by reaction of γ-butyrolactone;
(e) passing the 1, 4˜butaendiol product stream from step (d) to a second distillation column operated such that (4-hyroxybutyl)-4-hydroxybutyrate is removed as a bottom stream and removing a 1,4-butanediol stream as overhead; and (f) passing the overhead stream removed in (e) to a third distillation column and recovering a purified 1,4-butanediol stream.

Prior art such as WO2006037957A1 discloses that the amount of 2-(4′-hydroxybutoxy)-tetrahydrofuran and its precursors in at least one C₄ compound can be measured using the Peak Acetal Test. The Peak Acetal Test is disclosed as involving the removal of lights from the 1,4-butanediol crude hydrogenation product at 120° C. and then further heating at 160° C. for three hours. The heating which is carried out using an isomantle heater, round bottom flask, condenser and collection pot is carried out under a blanket of nitrogen at atmospheric pressure. The procedure allows for the reaction of the precursors of the acetal and is therefore disclosed in the prior art as reporting the maximum acetal content possible in the product 1,4-butandiol stream if the crude hydrogenation product was subjected to purification by a standard distillation system. The residue is then analysed by gas chromatography.

WO9736846A1 suggests that the butane-1,4-diol produced by the hydrogenolysis route of U.S. Pat. Nos. 4,584,419, 4,751,334 or WO88/00937 typically contains from about 0.15% by weight to about 0.20% by weight of the cyclic acetal.

WO2006037957A1 contains the same disclosure, but discloses Peak Acetal Tests on crude hydrogenation streams of 0.429 wt % and higher, with reductions to around 0.2 wt % following treatment of the streams according to that invention.

WO2013034881A1 does not address 2-(4′-hydroxybutoxy)-tetrahydrofuran levels.

WO2013076747A1 in the name of Conser SpA discloses a process for the production of 1,4-butanediol and tetrahydrofuran by catalytic hydrogenation of dialkyl maleates. The process consists essentially in the following steps:
a) hydrogenating a stream of dialkyl maleate in a first stage of reaction over suitable catalysts to produce dialkyl succinate;
b) further hydrogenating the dialkyl succinate in a second stage of reaction, by using a different suitable catalyst, for producing mainly 1,4-butanediol, together with gamma-butyrolactone and tetrahydrofuran as co-products.

In both stages of reaction the conditions, as hydrogen/organic feed ratio, pressure and temperature, are such to maintain the reactors in mixed liquid/vapor phase.

WO2013076747A1 also discloses that, to an extent surprisingly more favourable than expected, the tests produced using the WO2013076747A1 reaction described above in mixed phase and in two steps showed that the formation of the by-product cyclic acetal, the 2-(hydroxybutoxy)-tetrahydrofuran, which WO2013076747A1 states represents a particularly undesired impurity due to its boiling point very close to that of BDO, is considerably reduced compared to other similar processes in vapor phase. Note that the 2-(hydroxybutoxy)-tetrahydrofuran to which WO2013076747A1 refers is the same cyclic acetal as that referred to as 2-(4′-hydroxybutoxy)-tetrahydrofuran in the other prior art and the rest of this document. WO2013076747A1 states this reduction in by-product cyclic acetal formation represents a further and not negligible advantage of the WO2013076747A1 invention, contrasting it to US2007/0260073 (corresponding to WO2006037957A1). WO2013076747A1 states that US2007/0260073 teaches that the reduction of acetal may be achieved by contacting in liquid phase with a stream of hydrogen and in presence of catalysts of the same type described in the WO2013076747A1 invention the butanediol produced, normally in vapor phase, in another hydrogenolysis reactor. WO2013076747A1 states that the WO2013076747A1 invention reaches a still better result, in terms of acetal contamination, simply by operating the hydrogenolysis reaction in mixed liquid-gas phase, without any need of additional purification step in liquid phase. In other words, WO2013076747A1 is saying that the mixed liquid gas-phase hydrogenolysis disclosed in WO2013076747A1 means a polishing hydrogenation as in US2007/0230073 is not required because of reduced by-product cyclic acetal formation in the mixed liquid-gas phase hydrogenolysis.

The applicant has however surprisingly found that, even when there are low levels of the cyclic acetal 2-(4′-hydroxybutoxy)-tetrahydrofuran in the crude 1,4-butanediol stream produced by hydrogenolysis of dialkyl succinate, there may still be unacceptably high levels in the purified 1,4-butanediol after refining to remove alkanol and valuable by-products such as γ-butyrolactone and tetrahydrofuran.

Preferred embodiments of the present invention seek to overcome one or more of the above disadvantages of the prior art. In particular, preferred embodiments of the present invention seek to provide improved processes of producing refined 1,4-butanediol with low levels of the cyclic acetal 2-(4′-hydroxybutoxy)-tetrahydrofuran.

### SUMMARY OF INVENTION

According to a first aspect of the invention, there is provided a process for producing a refined 1,4-butanediol stream, the process comprising hydrogenolysis of dialkyl succinate in one or more mixed vapour/liquid phase reaction stages to form a crude 1,4-butanediol stream comprising 1,4-butanediol, γ-butyrolactone, tetrahydrofuran and alkanol and passing the crude 1,4-butanediol stream to a refining process, wherein at least some of the γ-butyrolactone, tetrahydrofuran and alkanol is removed from the 1,4-butanediol, and recovering from the refining process a refined 1,4-butanediol stream having a higher concentration of 1,4-butanediol than the crude 1,4-butanediol stream, wherein the refining process comprises a polishing section in which an intermediate stream comprising 1,4-butanediol and 2-(4′-hydroxybutoxy)-tetrahydrofuran is passed over a catalytic bed to reduce the 2-(4′-hydroxybutoxy)-tetrahydrofuran content of the intermediate stream.

A particular process in which the present invention may be advantageous is a process involving a mixed-phase hydrogenolysis, for example a mixed phase hydrogenolysis as disclosed in WO2013076747A1. Such mixed phase hydrogenolysis processes have been asserted in the prior art to be beneficial because of the low 2-(4′-hydroxybutoxy)-tetrahydrofuran level in the hydrogenolysis product. However, the applicant has found that further 2-(4′-hydroxybutoxy)-tetrahydrofuran may be produced during refining of the 1,4-butanediol and that the process of the present invention is therefore a valuable addition to a mixed phase hydrogenolysis process.

The dialkyl succinate may be produced by hydrogenation of dialkyl maleate. Preferably, the hydrogenation of dialkyl maleate to dialkyl succinate is carried out in one or more separate reaction stages upstream of the hydrogenolysis. Preferably different catalysts are used for the hydrogenation of dialkyl maleate to dialkyl succinate and the hydrogenolysis of dialkyl succinate to produce the crude 1,4-butanediol stream. Preferably the reaction stages are mixed vapour/liquid phase reaction stages. That is, the conditions in the reaction stage are such as to maintain a mixed liquid/vapour phase. That may be achieved for example by controlling one or more of conditions such as the feed ratio of hydrogen to organic feed, the pressure and the temperature. In some embodiments however the hydrogenation of dialkyl maleate to dialkyl succinate may be carried out in the same one or more reaction stages as the hydrogenolysis of dialkyl succinate. In such embodiments, dialkyl maleate may be fed to the one or more reaction stages where the dialkyl maleate undergoes hydrogenation to dialkyl succinate that then undergoes hydrogenolysis in the same reaction zone to form 1,4-butanediol, typically together with co-products γ-butyrolactone and tetrahydrofuran. The dialkyl succinate may thus be seen as an intermediate in the reaction stages. Preferably the reaction stages are mixed vapour/liquid phase reaction stages. That is, the conditions in the reaction stage are such as to maintain a mixed liquid/vapour phase. That may be achieved for example by controlling one or more of conditions such as the feed ratio of hydrogen to organic feed, the pressure and the temperature.

For example, a mixed phase hydrogenation and hydrogenolysis of dialkyl maleate to 1,4-butanediol may comprise a first reaction stage in which dialkyl maleate is hydrogenated over a catalyst to produce dialkyl succinate and a second reaction stage in which the dialkyl succinate is further hydrogenated to 1,4-butanediol, typically together with co-products γ-butyrolactone and tetrahydrofuran. Preferably the second reaction stage is carried out over a different catalyst to the first reaction stage, although in some embodiments the catalysts may be the same. For example the catalyst in the first reaction stage may comprise palladium, for example supported on a support comprising carbon or alumina. For example, the catalyst in the second reaction stage may comprise copper, such as a copper-chromite catalyst or a copper-zinc oxide catalyst. In each reaction stage the conditions are such as to maintain a mixed liquid/vapour phase. That may be achieved by controlling one or more of conditions such as the feed ratio of hydrogen to organic feed, the pressure and the temperature. Preferably the first reaction stage is carried out in a first reactor and the second reaction stage is carried out in a second reactor, although in some embodiments the two stages may be carried out in a single reactor, for example having two or more zones.

A source of hydrogen, typically hydrogen gas, will typically be added to the reaction stages in which the hydrogenolysis takes place.

Preferably the catalytic bed in the polishing section comprises a catalyst comprising an active metal. The active metal may comprise a platinum group metal. The active metal may comprise nickel or copper. Preferably the active metal comprises at least one of nickel, copper, palladium, platinum, rhodium and ruthenium. Preferably the catalyst comprises a support. Preferably the support comprises alumina, silica, zirconia, zinc, chromium, carbon or mixtures thereof such as silica/alumina or zirconia/alumina. In some instances, silica may be added to the support, which may improve the hydrothermal stability of the support.

Preferably the intermediate stream is contacted with hydrogen over the catalytic bed. The hydrogen may be introduced, for example, as a hydrogen gas stream.

Preferably the hydrogen pressure in the catalytic bed is from 20 barg to 60 barg, more preferably from 30 barg to 50 barg, most preferably around 40 barg. Preferably the temperature in the catalytic bed is from 40° C. to 160° C., more preferably 80° C. to 120° C. Preferably the intermediate stream further comprises water or alkanol, most preferably water, and the intermediate stream is contacted with hydrogen over the catalytic bed. Preferably the water or alkanol is in an amount of from 0 wt % to 30 wt %, more preferably from 1 wt % to 30 wt %, yet more preferably from 5 wt % to 30 wt %, even more preferably from 5 wt % to 20 wt % and most preferably from 10 wt % to 20 wt % of the intermediate stream.

Thus, advantageously, the 2-(4′-hydroxybutoxy)-tetrahydrofuran undergoes hydrolysis then hydrogenation. Water is most preferred as the 2-(4′-hydroxybutoxy)-tetrahydrofuran then advantageously undergoes hydrolysis then hydrogenation to recover 2 moles of 1,4-butanediol per mole of 2-(4′-hydroxybutoxy)-tetrahydrofuran. Water may then be removed by distillation and recycled.

Preferably the polishing section comprises a trickle bed; that is, the catalytic bed is a trickle bed, and more preferably a reactor comprising multiple catalytic beds with flow distributors between each catalytic bed.

Preferably the intermediate stream comprises an acid. The acid may be added directly to the intermediate stream.

However, preferably the acid is added by adding an acid-forming species to the intermediate stream or, most preferably, by not, or not fully, removing an acid-forming species from the intermediate stream. Preferably the acid-forming species is γ-butyrolactone. For example, while the refining process may separate the γ-butyrolactone that is in the crude 1,4-butanediol stream as a γ-butyrolactone product, a portion of the γ-butyrolactone may remain unseparated and be comprised in the intermediate stream. The acid may advantageously promote the hydrolysis of the acetal, 2-(4′-hydroxybutoxy)-tetrahydrofuran, to a hemi-acetal, which may then undergo hydrogenation more quickly than the acetal itself. The presence of the acid may thus remove the hydrolysis as a rate-limiting step and improve the overall reaction rate. Forming the acid from γ-butyrolactone may be advantageous as no separate acid stream is then needed to add acid into to the process.

Preferably the polishing section is located towards a downstream end of the refining process. In that way, the polishing section preferably removes 2-(4′-hydroxybutoxy)-tetrahydrofuran formed in the hydrogenolysis or from precursors formed in the hydrogenolysis, and also any 2-(4′-hydroxybutoxy)-tetrahydrofuran formed in other ways in the refining process. 2-(4′-hydroxybutoxy)-tetrahydrofuran may be formed for example due to ingress of air into the refining process, particularly into vacuum columns in the refining process, or by dehydrogenation of 1,4-butanediol which can be catalysed in the presence of fines from the hydrogenolysis of the dialkyl succinate.

Preferably the refining process comprises at least one vacuum distillation column and the polishing section is located downstream of the at least one vacuum distillation column.

In one embodiment, the polishing section may be the last unit in the refining process. Such an embodiment may be advantageous where a polishing section is retrofitted to an existing refining process that does not include a polishing section.

In one embodiment the crude 1,4-butanediol stream may be passed to a crude column preferably operated such that the tetrahydrofuran is withdrawn in an overhead stream and the γ-butyrolactone and 1,4-butanediol are withdrawn in a bottoms stream. The overhead stream is preferably passed to a THE column or columns from which purified tetrahydrofuran is recovered, preferably with a recycle of tetrahydrofuran and alkanol to the crude column. Preferably the bottoms stream is passed to a lights column, in which alkanol is preferably removed in an overhead stream, with the γ-butyrolactone and 1,4-butanediol being preferably withdrawn in a further bottoms stream that is passed to a heavies column. In the heavies column, γ-butyrolactone and dimethyl-succinate are preferably taken overhead, with 1,4-butanediol preferably withdrawn as a 1,4-butanediol side draw and heavies preferably removed as a heavies bottom stream. One or both of the lights column and heavies column are preferably vacuum distillation columns. The γ-butyrolactone is preferably purified via a DMS recycle column, that separates the dimethyl-succinate for recycle from the γ-butyrolactone, and a γ-butyrolactone heavies column in which remaining heavy components are preferably removed to form a refined γ-butyrolactone stream, typically taken as a side draw from the Y-butyrolactone heavies column. The 1,4-butanediol side draw is preferably passed to the polishing section for removal of the 2-(4′-hydroxybutoxy)-tetrahydrofuran. A polished 1,4-butanediol stream recovered from the polishing section is preferably passed to a BDO product column from which the refined 1,4-butanediol is taken, preferably as a side draw. In some embodiments the side draw may be passed to a side-stripper column to produce the refined 1,4-butanediol.

Preferably the polishing section comprises adding water, or in some embodiments an alkanol, to the intermediate stream before passing the intermediate stream to a polishing reactor along with a stream comprising hydrogen. A polishing reactor effluent stream having reduced 2-(4′-hydroxybutoxy)-tetrahydrofuran content compared to the intermediate stream is withdrawn from the polishing reactor. Water is preferably removed from the polishing reactor effluent stream, preferably in a water stripper column, and preferably recycled. After water removal, a polished 1,4-butanediol stream is obtained having a lower 2-(4′-hydroxybutoxy)-tetrahydrofuran content than the intermediate stream.

In an embodiment of the polishing section, the intermediate stream is preferably fed to a feed drum in which it is mixed with water. From the feed drum, the intermediate stream is preferably fed to a polishing reactor preferably comprising a trickle catalytic bed and more preferably comprising multiple catalytic beds with flow distributors between each catalytic bed. A stream comprising a source of hydrogen, preferably hydrogen gas, is also preferably fed to the polishing reactor. The intermediate stream and the stream comprising hydrogen gas are preferably both fed at or near the top of the polishing reactor. For example, both streams may be fed to a headspace of the polishing reactor or above an uppermost catalyst bed in the polishing reactor. A polishing reactor effluent stream, having a reduced content of 2-(4′-hydroxybutoxy)-tetrahydrofuran is withdrawn from the polishing reactor. The polishing reactor effluent stream is preferably withdrawn at or near the bottom of the reactor, for example from below a lowermost catalyst bed in the polishing reactor. The polishing reactor effluent stream is preferably passed to a knock-out drum, preferably after passing through a filter. From the knock-out drum, a liquid stream is preferably fed to a water stripper column from which a polished 1,4-butanediol stream is withdrawn, preferably from the bottom of the water stripper column. An overhead stream from the water stripper column preferably comprises water, which is preferably condensed and recycled to the feed drum.

It may be that the 2-(4′-hydroxybutoxy)-tetrahydrofuran concentration in the intermediate stream fed to the polishing section is at least 1.5 times, preferably at least double, the 2-(4′-hydroxybutoxy)-tetrahydrofuran concentration in the crude 1,4-butanediol stream from the hydrogenolysis of dialkyl-succinate. Thus, while the acetal level in the crude 1,4-butanediol stream may appear acceptable, additional 2-(4′-hydroxybutoxy)-tetrahydrofuran produced in the refining process may raise the acetal level such that, without the polishing section of the present invention, the refined 1,4-butanediol stream would have too high an acetal level. The present invention may thus offer a significant advantage in processes that would not be expected to require acetal removal downstream of the hydrogenolysis of dialkyl-succinate.

Preferably the refined 1,4-butanediol stream comprises less than 0.15 wt %, more preferably less than 0.1 wt %, more preferably less than 0.8 wt %, more preferably less than 0.6 wt % and more preferably less than 0.4 wt % 2-(4′-hydroxybutoxy)-tetrahydrofuran.

The dialkyl succinate preferably comprises C₁-C₆ alkyl groups and more preferably C₁-C₄ alkyl groups. Particularly preferred are dimethyl succinate and diethyl succinate and more preferably dimethyl succinate. The dialkyl maleate preferably comprises C₁-C₆ alkyl groups and more preferably C₁-C₄ alkyl groups. Particularly preferred are dimethyl maleate and diethyl maleate and more preferably dimethyl maleate.

While the present invention may be particularly advantageous with mixed phase hydrogenolysis processes, which the prior art has maintained do not require polishing sections, the present invention may equally be applicable to other hydrogenolysis methods that appear to produce a low level of 2-(4′-hydroxybutoxy)-tetrahydrofuran in the crude 1,4-butanediol stream. The inventors have appreciated that 2-(4′-hydroxybutoxy)-tetrahydrofuran is created not just as a by-product of the hydrogenolysis, or as a result of precursors formed as a by-product of the hydrogenolysis, but is also formed from extra reactions taking place in the refining process. Thus, even if the 2-(4′-hydroxybutoxy)-tetrahydrofuran level in the crude 1,4-butanediol stream leaving the hydrogenolysis appears to be already acceptably low, a polishing section according to the present invention is still advantageous to treat new 2-(4′-hydroxybutoxy)-tetrahydrofuran formed in the refining process. Thus, according to a second aspect of the invention there is provided a process for producing a refined 1,4-butanediol stream, the process comprising hydrogenolysis of dialkyl succinate to form a crude 1,4-butanediol stream comprising 1,4-butanediol, γ-butyrolactone, tetrahydrofuran, alkanol and less than 0.15 wt % 2-(4′-hydroxybutoxy)-tetrahydrofuran and passing the crude 1,4-butanediol stream to a refining process, wherein at least some of the γ-butyrolactone, tetrahydrofuran and alkanol is removed from the 1,4-butanediol, and recovering from the refining process a refined 1,4-butanediol stream having a higher concentration of 1,4-butanediol than the crude 1,4-butanediol stream, wherein the refining process comprises a polishing section in which an intermediate stream comprising 1,4-butanediol and 2-(4′-hydroxybutoxy)-tetrahydrofuran is passed over a catalytic bed to reduce the 2-(4′-hydroxybutoxy)-tetrahydrofuran content of the intermediate stream.

Preferably the crude 1,4-butanediol stream comprises less than 0.1 wt % and more preferably less than 0.75 wt % 2-(4′-hydroxybutoxy)-tetrahydrofuran. It may be that the crude 1,4-butanediol stream comprises less than 0.7 wt %, or less than 0.6 wt %, or less than 0.5 wt %, or less than 0.4 wt %, or less than 0.3 wt %, or less than 0.2 wt % 2-(4′-hydroxybutoxy)-tetrahydrofuran. Such low levels would suggest on their face that there would not be an issue with 2-(4′-hydroxybutoxy)-tetrahydrofuran levels in the final product, but the inventors have appreciated that new 2-(4′-hydroxybutoxy)-tetrahydrofuran may be formed in the refining process and that a refining process comprising a polishing section according to the present invention may thus still be advantageous.

It may be that the 2-(4′-hydroxybutoxy)-tetrahydrofuran concentration in the intermediate stream is at least 1.5 times, preferably at least double, the 2-(4′-hydroxybutoxy)-tetrahydrofuran concentration in the crude 1,4-butanediol stream from the hydrogenolysis of dialkyl-succinate. For example, the intermediate stream may comprise at least 0.2 wt %, more preferably at least 0.25 wt %, and yet more preferably at least 0.3 wt %, 2-(4′-hydroxybutoxy)-tetrahydrofuran.

Preferably the intermediate stream is contacted with hydrogen over the catalytic bed. The hydrogen may be introduced, for example, as a hydrogen gas stream.

Preferably the hydrogen pressure in the catalytic bed is from 20 barg to 60 barg, more preferably from 30 barg to 50 barg, most preferably around 40 barg. Preferably the temperature in the catalytic bed is from 40° C. to 160° C., more preferably 80° C. to 120° C.

Preferably the intermediate stream further comprises water or alkanol, most preferably water, and the intermediate stream is contacted with hydrogen over the catalytic bed. Preferably the water or alkanol is in an amount of from 0 wt % to 30 wt %, more preferably from 1 wt % to 30 wt %, yet more preferably from 5 wt % to 30 wt %, even more preferably from 5 wt % to 20 wt % and most preferably from 10 wt % to 20 wt % of the intermediate stream.

Preferably the intermediate stream comprises an acid. The acid may be added directly to the intermediate stream. However, preferably the acid is added by adding an acid-forming species to the intermediate stream or, most preferably, by not, or not fully, removing an acid-forming species from the intermediate stream. Preferably the acid-forming species is γ-butyrolactone. For example, while the refining process may separate the γ-butyrolactone that is in the crude 1,4-butanediol stream as a γ-butyrolactone product, a portion of the γ-butyrolactone may remain unseparated and be comprised in the intermediate stream.

Preferably the polishing section is located towards a downstream end of the refining process. In that way, the polishing section preferably removes 2-(4′-hydroxybutoxy)-tetrahydrofuran formed in the hydrogenolysis and also any 2-(4′-hydroxybutoxy)-tetrahydrofuran formed in the refining process. 2-(4′-hydroxybutoxy)-tetrahydrofuran may be formed in the refining process from precursors formed in the hydrogenolysis, but further 2-(4′-hydroxybutoxy)-tetrahydrofuran may also be formed in the refining process for example due to ingress of air into the refining process, particularly into vacuum columns in the refining process, or by dehydrogenation of 1,4-butanediol which can be catalysed in the presence of fines from the hydrogenolysis of the dialkyl succinate. At least some of such further 2-(4′-hydroxybutoxy)-tetrahydrofuran is advantageously removed in the polishing section.

Preferably the refining process comprises at least one vacuum distillation column and the polishing section is located downstream of the at least one vacuum distillation column.

The amount of 2-(4′-hydroxybutoxy)-tetrahydrofuran is measured using the Peak Acetal Test described above. The Peak Acetal Test involves the removal of lights from the stream to be measured at 120° C. and then further heating at 160° C. for three hours. The heating, which may be carried out using an isomantle heater, round bottom flask, condenser and collection pot, is carried out under a blanket of nitrogen at atmospheric pressure. The residue is then analysed by gas chromatography to determine the 2-(4′-hydroxybutoxy)-tetrahydrofuran content. The procedure allows for the reaction of precursors of 2-(4′-hydroxybutoxy)-tetrahydrofuran in the stream. In an industrial 1,4-butanediol plant, the precursors will react to form 2-(4′-hydroxybutoxy)-tetrahydrofuran by the time the refined 1,4-butanediol stream is recovered and the Peak Acetal Test therefore measures the amount of 2-(4′-hydroxybutoxy)-tetrahydrofuran to be expected in a final 1,4-butanediol product that is attributable to 2-(4′-hydroxybutoxy)-tetrahydrofuran or precursors already present in the stream being measured. In the prior art, the test has sometimes been suggested to therefore represent the maximum 2-(4′-hydroxybutoxy)-tetrahydrofuran content possible in the final product 1,4-butandiol stream if the crude hydrogenolysis product was subjected to purification by a standard distillation system. However, as set out above, the inventors have appreciated that new 2-(4′-hydroxybutoxy)-tetrahydrofuran is formed during the refining process, for example due to air ingress or catalyst fines, and that even when the Peak Acetal Test reports 2-(4′-hydroxybutoxy)-tetrahydrofuran levels in a crude 1,4-butanediol stream that would appear to be acceptable, the final product can contain unacceptable 2-(4′-hydroxybutoxy)-tetrahydrofuran levels unless the present invention is employed.

The process of the second aspect of the invention may additionally or alternatively include any features described above, for example in relation to the first aspect of the invention.

It will be appreciated that features described in relation to one aspect of the invention may be equally applicable in another aspect of the invention. For example, features described in relation to the first aspect of the invention, may be equally applicable to the second aspect of the invention, and vice versa. Some features may not be applicable to, and may be excluded from, particular aspects of the invention.

### DESCRIPTION OF THE DRAWINGS

Embodiments of the present invention will now be described, by way of example, and not in any limitative sense, with reference to the accompanying drawings, of which:

FIG. 1 is an illustration diagram of a process including an embodiment of the invention;

FIG. 2 is an illustration of a polishing section suitable for use in the process of FIG. 1; and

FIG. 3 is a graph of Experiments 1, 2 3.

### DETAILED DESCRIPTION

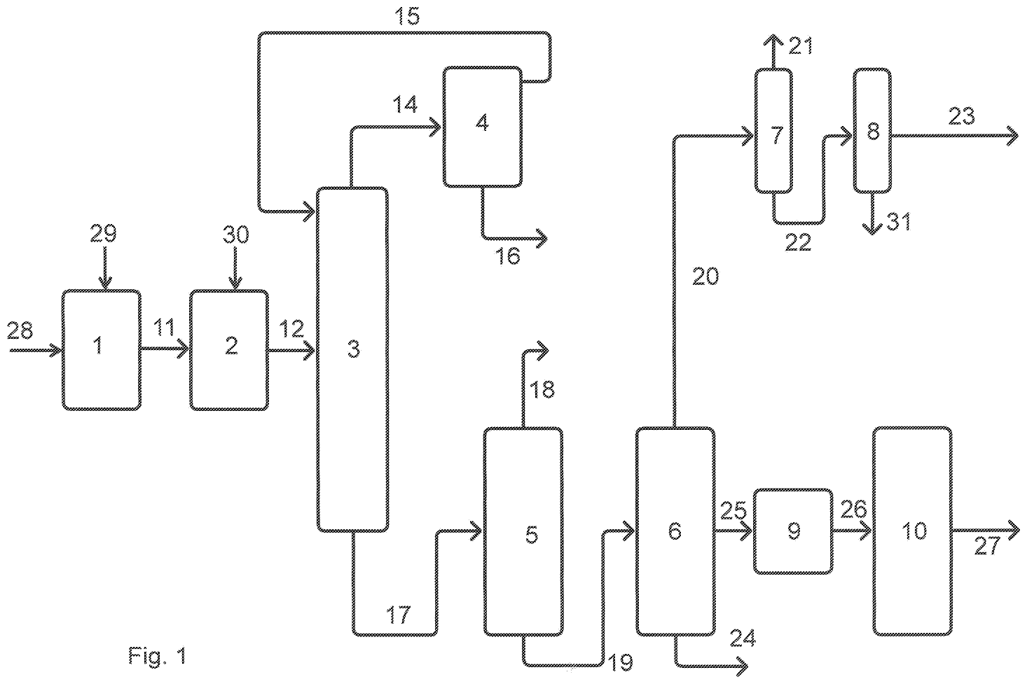

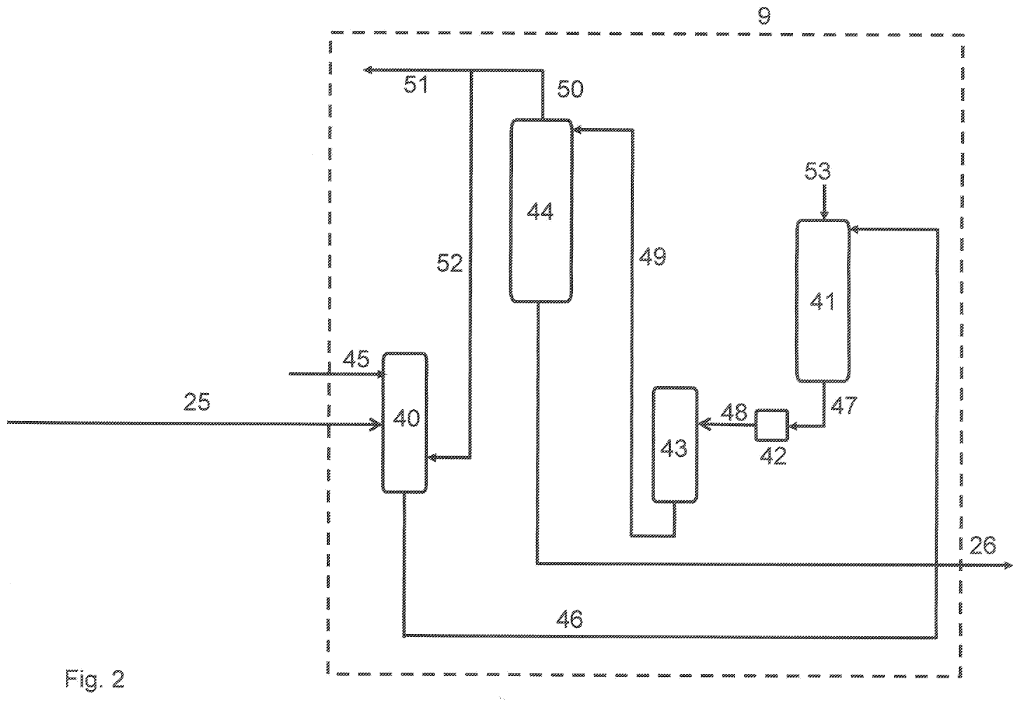

In FIG. 1 a feed of dialkyl maleate is supplied to a first reactor 1, to which hydrogen 29 is also supplied. At least some of the dialkyl maleate is hydrogenated to dialkyl succinate in the first reactor 1. A stream 11 comprising the dialkyl succinate is withdrawn from first reactor 1 and fed to second reactor 2, to which further hydrogen 30 is also supplied. In the second reactor 2, at least some of the dialkyl succinate is converted to 1,4-butanediol by hydrogenolysis. In this embodiment, both reactors 1 and 2 operate in a mixed vapour/liquid phase, but in other embodiments they may operate in the vapour phase or in the liquid phase. In other embodiments there may be only one reactor. In such embodiments the feed to the one reactor may comprise dialkyl succinate, for example produced from succinic acid, or it may comprise dialkyl maleate, for example produced form maleic anhydride. If it comprises dialkyl maleate the reactions in the one reactor will include both the hydrogenation of the dialkyl maleate to dialkyl succinate and the subsequent hydrogenolysis of the dialkyl succinate to 1,4-butanediol. In other embodiments, hydrogen may be fed to only the first reactor 1, or the hydrogen fed to the second reactor 2 may be at least partially hydrogen recovered from the first reactor 1. A crude 1,4-butanediol stream 12 comprising 1,4-butanediol, γ-butyrolactone, tetrahydrofuran and alkanol is recovered from the second reactor 2 and passed to a refining process. The crude 1,4-butanediol stream 12 is fed to a crude column 3 operated such that the tetrahydrofuran is withdrawn in an overhead stream 14 and the γ-butyrolactone and 1,4-butanediol are withdrawn in a bottoms stream 17. The overhead stream 14 is passed to a THE separation unit 4, typically comprising one or more columns, from which purified tetrahydrofuran 16 is recovered, with a recycle 15 of tetrahydrofuran and alkanol to the crude column 3. The bottoms stream 17 is passed to a lights column 5, in which alkanol is removed in an overhead alkanol stream 18, with the γ-butyrolactone and 1,4-butanediol being withdrawn in a further bottoms stream 19 that is passed to a heavies column 6. In the heavies column 6, γ-butyrolactone and dimethyl-succinate are taken overhead in a crude γ-butyrolactone stream 20, with 1,4-butanediol withdrawn as a 1,4-butanediol side draw 25 and heavies removed as a heavies bottom stream 24. In this embodiment the lights column 5 and heavies column 6 are vacuum distillation columns. The γ-butyrolactone in the crude γ-butyrolactone stream 20 is purified via a DMS recycle column 7, that separates the dimethyl-succinate into a DMS recycle 21, and a γ-butyrolactone heavies column 8 in which remaining heavy components are removed as a removed heavies stream 31. A refined γ-butyrolactone stream 23, is taken as a side draw from the γ-butyrolactone heavies column 8. The 1,4-butanediol side draw 25 is passed as a feed stream to a polishing section 9 in which an intermediate stream, which comprises 1,4-butanediol and 2-(4′-hydroxybutoxy)-tetrahydrofuran, is passed over a catalytic bed for removal of the 2-(4′-hydroxybutoxy)-tetrahydrofuran. An example embodiment of a suitable polishing section 9 is described below and in FIG. 2. A polished 1,4-butanediol stream 26 recovered from the polishing section 9 is passed to a BDO product column 10 from which a refined 1,4-butanediol stream 27 is taken as a side draw. In some embodiments the side draw may be passed to a side-stripper column to produce the refined 1,4-butanediol. Alternatively in some embodiments there may be a 1,4-butanediol pre-column before the BDO product column 10. The refined 1,4-butanediol stream 27 has a higher concentration of 1,4-butanediol than the crude 1,4-butanediol stream 12. The 1,4-butanediol side draw 25 has a higher 2-(4′-hydroxybutoxy)-tetrahydrofuran content than the crude 1,4-butanediol stream 12 because 2-(4′-hydroxybutoxy)-tetrahydrofuran is formed in one or more of the crude column 3, the lights column 5 and the heavies column 6. The polishing section 9 advantageously reduces the 2-(4′-hydroxybutoxy)-tetrahydrofuran content back to an acceptable level so that the refined 1,4-butanediol stream 27 has a 2-(4′-hydroxybutoxy)-tetrahydrofuran concentration that is lower than that in the 1,4-butanediol side draw 25 and that meets the specification for the desired downstream use of the 1,4-butanediol. In the embodiment of FIG. 1 the polishing section 9 is before the BDO product column 10. However, the polishing section 9 may be at other points in the refining process and may, for example be downstream of the BDO product column 10. Such an arrangement may be particularly advantageous where the polishing section 9 is retrofitted to a plant.

An embodiment of a polishing section 9 suitable for use in the process described above in relation to FIG. 1 is illustrated in FIG. 2. It will be appreciated that other arrangements of the polishing section 9 are also possible. In FIG. 2, the 1,4-butanediol side draw 25, which comprises 1,4-butanediol and 2-(4′-hydroxybutoxy)-tetrahydrofuran, is fed as a feed stream to the polishing section 9. In the polishing section 9, the 1,4-butanediol side draw 25 is fed to a feed drum 40 in which it is mixed with a water stream 45. An intermediate stream 46 from the feed drum 40 is fed to a polishing reactor 41 comprising multiple catalyst beds with flow distributors between each bed. A hydrogen feed stream 53 comprising hydrogen gas is also fed to the polishing reactor 41. The intermediate stream 46 from the feed drum 40 and the hydrogen feed stream 53 are both fed near the top of the polishing reactor 41 above an uppermost catalyst bed in the polishing reactor 41. A polishing reactor effluent stream 47, having a reduced content of 2-(4′-hydroxybutoxy)-tetrahydrofuran, is withdrawn near the bottom of the polishing reactor 41, below a lowermost catalyst bed in the polishing reactor 41. The polishing reactor effluent stream 42 is passed through a filter 42 to form a filtered polishing reactor effluent stream 48, which is passed to a knock-out drum 43. From the knock-out drum 43, a liquid stream 49 is fed to a water stripper column 44 from the bottom of which the polished 1,4-butanediol stream 26 is withdrawn. The polished 1,4-butanediol stream 26 has a lower 2-(4′-hydroxybutoxy)-tetrahydrofuran content than the 1,4-butanediol side draw 25. An overhead water stream 50 from the water stripper column 44 is condensed and recycled 52 to the feed drum 40, with a purge 51 that is recycled to a point upstream in the refining process.

### Experiments

### Experiment 1

To a 500 ml reaction vessel was charged 400 g of crude hydrogenation product. The reaction vessel was heated under an inert atmosphere of nitrogen to 120° C. and the light components were removed by distillation. The unit was then modified to operate in reflux mode to ensure no further losses could occur and the temperature increased to 160° C., at which point samples were taken with time and analysed for acetal content by GC.

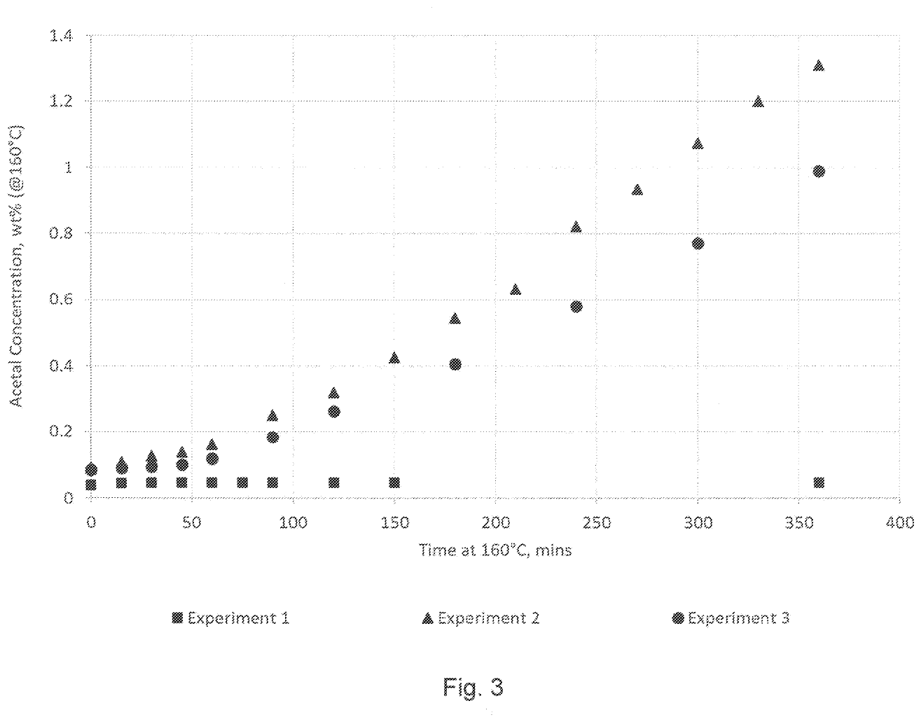

The maximum 2-(4′-hydroxybutoxy)-tetrahydrofuran level for this system was found to be 1800 ppm. A graphical summary of this experiment can be found in FIG. 3.

### Experiment 2

Experiment 1 was repeated with the exception that air was used instead of nitrogen. The results indicated that, over the first 45 minutes of the test the rate of 2-(4′-hydroxybutoxy)-tetrahydrofuran formation was consistent with that when nitrogen was used. However, as the initial acetal precursors were consumed as shown by the first experiment, the presence of air resulted in significant increases in acetal as the test proceeded. A summary of this experiment, as compared to experiment 1, can be found in FIG. 3.

### Experiment 3

Experiment 1 was repeated with the exception that approximately 10 wt % of powdered hydrogenation catalyst was added to the reactor. Analysis of samples with time suggests that high levels of catalyst fines promoted the formation of 2-(4′-hydroxybutoxy)-tetrahydrofuran. A summary of this experiment, as compared to experiment 1, can be found in FIG. 3.

The experiments demonstrate two mechanisms by which 2-(4′-hydroxybutoxy)-tetrahydrofuran levels may increase during refining of 1,4-butanediol product. As a result, even crude 1,4-butanediol streams that appear to have acceptably low levels of 2-(4′-hydroxybutoxy)-tetrahydrofuran, including when measured in a Peak Acetal Test, may surprisingly result in excessive 2-(4′-hydroxybutoxy)-tetrahydrofuran in the refined 1,4-butanediol stream. The present invention mitigates this problem by including a polishing section in which an intermediate stream comprising 1,4-butanediol and 2-(4′-hydroxybutoxy)-tetrahydrofuran is passed over a catalytic bed to reduce the 2-(4′-hydroxybutoxy)-tetrahydrofuran content. The inclusion of such a polishing section is counter-intuitive when the crude 1,4-butanediol stream appears to have an acceptably low level of 2-(4′-hydroxybutoxy)-tetrahydrofuran, not least because adding extra, apparently unnecessary, equipment may increase capital and operating costs. However, the applicant has appreciated that, as demonstrated by experiments 1 to 3,2-(4′-hydroxybutoxy)-tetrahydrofuran levels can increase during refining, and the inclusion of the polishing section of the present invention may therefore be advantageous. For example, the cost of the polishing section may be favourable when compared with the cost of measures required to ensure zero unwanted influx of air or fines into the refining process.

It will be appreciated by persons skilled in the art that the above embodiments have been described by way of example only, and not in any limitative sense, and that various alterations and modifications are possible without departure from the scope of the invention as defined by the appended claims. For example, different orders of the separations, or different numbers of columns are possible in the refining process, and the location of the polishing section within the refining process may be altered.

1. A process for producing a refined 1,4-butanediol stream, the process comprising hydrogenolysis of dialkyl succinate in one or more mixed vapour/liquid phase reaction stages to form a crude 1,4-butanediol stream comprising 1,4-butanediol, γ-butyrolactone, tetrahydrofuran and alkanol and passing the crude 1,4-butanediol stream to a refining process, wherein at least some of the γ-butyrolactone, tetrahydrofuran and alkanol is removed from the 1,4-butanediol, and recovering from the refining process a refined 1,4-butanediol stream having a higher concentration of 1,4-butanediol than the crude 1,4-butanediol stream, wherein the refining process comprises a polishing section in which an intermediate stream comprising 1,4-butanediol and 2-(4′-hydroxybutoxy)-tetrahydrofuran is passed over a catalytic bed to reduce the 2-(4′-hydroxybutoxy)-tetrahydrofuran content of the intermediate stream.

2. A process according to claim 1, wherein the intermediate stream further comprises an alkanol or water and wherein the intermediate stream is contacted with hydrogen over the catalytic bed.

3. A process according to claim 2, wherein the hydrogen pressure in the catalytic bed is from 20 barg to 60 barg.

4. A process according to claim 2, wherein the intermediate stream further comprises an acid.

5. A process according to claim 4 wherein the acid is formed from γ-butyrolactone in the refining process.

6. A process according to claim 1, wherein the dialkyl succinate is produced by hydrogenation of dialkyl maleate.

7. A process according to claim 6, wherein the hydrogenation takes place in one or more separate mixed vapour/liquid phase reaction stages.

8. A process according to claim 1, wherein the temperature in the catalytic bed is from 40° C. to 160° C.

9. A process according to claim 1 wherein the catalytic bed comprises a catalyst comprising an active metal, preferably comprising at least one of nickel, copper, palladium, platinum, rhodium and ruthenium.

10. A process according to claim 9 wherein the catalyst comprises a support, preferably comprising alumina, silica, zirconia, zinc, chromium, carbon or mixtures thereof.

11. A process according to claim 1 wherein the polishing section is located towards a downstream end of the refining process so as to remove 2-(4′-hydroxybutoxy)-tetrahydrofuran formed in the refining process.

12. A process according to claim 1 wherein the refining process comprises at least one vacuum distillation column and the polishing section is located downstream of the at least one vacuum distillation column.

13. A process according to claim 1 wherein the polishing section comprises mixing a feed stream comprising 1,4-butanediol and 2-(4′-hydroxybutoxy)-tetrahydrofuran with water to form the intermediate stream and passing the intermediate stream, along with a stream comprising hydrogen, to a polishing reactor comprising the catalytic bed.

14. A process according to claim 13 wherein a polishing reactor effluent stream having reduced 2-(4′-hydroxybutoxy)-tetrahydrofuran content compared to the intermediate stream is withdrawn from the polishing reactor; and wherein water is removed from the polishing reactor effluent stream, preferably in a water stripper column, to obtain a polished 1,4-butanediol stream having a lower 2-(4′-hydroxybutoxy)-tetrahydrofuran content than the feed stream.

15. A process according to claim 13 wherein the 2-(4′-hydroxybutoxy)-tetrahydrofuran concentration in the feed stream is at least 1.5 times the 2-(4′-hydroxybutoxy)-tetrahydrofuran concentration in the crude 1,4-butanediol stream.

16. A process for producing a refined 1,4-butanediol stream, the process comprising hydrogenolysis of dialkyl succinate to form a crude 1,4-butanediol stream comprising 1,4-butanediol, γ-butyrolactone, tetrahydrofuran, alkanol and less than 0.15 wt % 2-(4′-hydroxybutoxy)-tetrahydrofuran and passing the crude 1,4-butanediol stream to a refining process, wherein at least some of the γ-butyrolactone, tetrahydrofuran and alkanol is removed from the 1,4-butanediol, and recovering from the refining process a refined 1,4-butanediol stream having a higher concentration of 1,4-butanediol than the crude 1,4-butanediol stream, wherein the refining process comprises a polishing section in which an intermediate stream comprising 1,4-butanediol and 2-(4′-hydroxybutoxy)-tetrahydrofuran is passed over a catalytic bed to reduce the 2-(4′-hydroxybutoxy)-tetrahydrofuran content of the intermediate stream.

17. A process according to claim 16 wherein the 2-(4′-hydroxybutoxy)-tetrahydrofuran concentration in the intermediate stream is at least 1.5 times the 2-(4′-hydroxybutoxy)-tetrahydrofuran concentration in the crude 1,4-butanediol stream.
